(19) United States
(12) Patent Application Publication
(10) Pub. No.: US 2012/0052056 A1
(43) Pub. Date: Mar. 1, 2012

(54) COMPOSITION FOR IMPROVING BLOOD CIRCULATION CONTAINING FERMENTED TEA, AND PHARMACEUTICAL AND HEALTH-FOOD COMPOSITIONS COMPRISING THE SAME

(76) Inventors: Hyun Jung Shin, Seoul (KR); Chae Wook Kim, Yongin-si (KR); Bum Jin Lee, Seoul (KR); Dae Bang Seo, Gyeonggi-do (KR)

(21) Appl. No.: 13/318,933

(22) PCT Filed: May 12, 2010
(86) PCT No.: PCT/KR2010/003018

§ 371 Date: Nov. 4, 2011

(30) Foreign Application Priority Data
May 12, 2009 (KR) 10-2009-0041263

(51) Int. Cl. A61K 36/064; A61P 9/10; A61P 3/04; A61P 3/06; A61P 3/10; A61P 25/00; A61P 9/12

(52) U.S. Cl. 424/115

(57) ABSTRACT

The present invention relates a composition containing, as an active ingredient, a post-fermented tea extract obtained by inoculating green tea with yeast as a fermentation strain and fermenting the inoculated green tea for a predetermined time. The composition has an excellent preference while it reduces the serum and liver cholesterol and triglyceride levels and has an excellent ability to relax blood vessels by inhibiting vasoconstriction, thus improving blood circulation. Due to its function to improve blood circulation, these compositions may be used as health foods, medical drugs and the like, which are effective against diseases, including stroke, myocardial infarction, hypertension, hyperlipidemia, diabetes or obesity.

### TECHNICAL FIELD

The present invention relates to a composition for improving blood circulation containing fermented tea, and pharmaceutical and health food compositions comprising the same.

### BACKGROUND ART

Green tea contains vitamins, caffeine, tannin, flavonoids, essential oil and the like (Yong-Ju LEE et al., Pharmacognosy, Dongmyung Publishing Co., 189-190, 1981). Green tea is generally known to show anti-diabetic, anti-obesity, antioxidant, anti-hypertension, antibacterial, cholesterol-lowering and antiulcer effects, and thus is usually taken as tea.

Such green tea is largely divided into unfermented tea and fermented tea, and the fermented tea can be sub-divided, according to the degree of fermentation, into mild fermented tea, semi-fermented tea, post-fermented tea, etc.

Typical examples of post-fermented tea include Pu-erh tea named after the Pu-erh region in China. The Pu-erh tea is post-fermented tea obtained from broad-leaf tea trees and refers to tea made by destroying the enzymes in the green tea leaves, and then depositing the green tea leaves to induce microbial propagation so as to ferment the green tea leaves.

Such pu-erh tea is known to lower blood pressure, reduce lipid levels and prevent arteriosclerosis. However, the pu-erh tea is not easy to take, because the leaves should be separated from the circular lumps. Also, it smells moldy and stale, because it is fermented by airborne bacteria. Moreover, it can be suspected of containing pathogenic microorganisms. In addition, it shows a low preference, because the first taste thereof is strongly astringent and is also bitter.

### DISCLOSURE

### Technical Problem

Therefore, it is an object of the present invention to provide a composition for improving blood comprising a fermented tea extract, which is easy to take, has an excellent preference and makes blood circulation smooth.

Another object of the present invention is to provide a pharmaceutical composition comprising said fermented tea extract.

Another object of the present invention is to provide a health food composition comprising said fermented tea extract.

### Technical Solution

To achieve the above objects, the present invention provides a composition for improving blood circulation containing, as an active ingredient, a fermented tea extract obtained by inoculating green tea with yeast as a fermentation strain.

The present invention also provides a pharmaceutical composition containing said fermented tea extract as an active ingredient.

The present invention also provides a health food composition containing said fermented tea extract as an active ingredient.

### Advantageous Effects

The composition for improving blood circulation comprising the fermented tea extract according to the present invention, and the pharmaceutical composition and health food composition comprising the same are prepared using yeast as a fermentation strain, so that these compositions have an excellent preference while they reduce the serum and liver cholesterol and triglyceride levels and have an excellent ability to relax blood vessels by inhibiting vasoconstriction, thus improving blood circulation. Due to their function to improve blood circulation, these compositions may be used as health foods, medical drugs and the like, which are effective against diseases, including stroke, myocardial infarction, hypertension, hyperlipidemia, diabetes or obesity.

### BRIEF DESCRIPTION OF DRAWINGS

FIG. 1 is a graphic diagram showing the free radical contents of reaction solutions treated with each of green tea, Pu-erh tea, post-fermented tea and vitamin C relative to a control containing no experimental sample.

### BEST MODE

A composition according to one embodiment of the present invention contains a fermented tea extract obtained by inoculating green tea with yeast and fermenting the inoculated green tea for a predetermined time. Examples of the yeast that is used in the present invention include Saccharomyces Carsbergensis, Saccharomyces Sake, Saccharomyces Ellipsoideus, Saccharomyces Coreanus, and Saccharomyces Cerevisiae. A composition according to one embodiment of the present invention may comprise a fermented tea extract obtained using at least one of the above-described yeasts as a fermentation strain. This fermented tea may be, for example, a post-fermented tea.

The fermentation process that uses the yeast as the fermentation strain is carried out at a temperature of 15˜30° C. for 24 hours to 28 days. Then, the green tea fermented thus is reflux-extracted with 70% ethanol for 12 hours, thereby obtaining a fermented tea extract.

The composition of the present invention contains the fermented tea extract in an amount of 0.1-50 wt % based on the total weight of the composition. If the content of the fermented tea extract in the composition is less than 0.1 wt %, the effect of improving blood circulation will not be shown, and if it is more than 50 wt %, an increase in the content thereof will not lead to a further increase in the effect thereof.

The fermented tea extract as described above has an excellent effect of improving blood circulation, and the composition comprising this fermented tea extract can be used as a pharmaceutical composition for preventing or treating stroke, myocardial infarction, hypertension, hyperlipidemia, diabetes or obesity by improving blood circulation. The composition of the present invention can be formulated into pills, capsules, tablets, granules, drinks and the like. The composition can generally be formulated using diluents or excipients, such as fillers, extenders, binders, wetting agents, disintegrants or surfactants. The excipient may be starch, calcium carbonate, sucrose, lactose or gelatin, and the formulation may include, in addition to a simple excipient, a lubricant such as magnesium stearate or talc.

Dosage units of the pharmaceutical composition may contain one-, two-, three- or four-fold amount of an individual dose, or ½, ⅓ or ¼-fold amount of an individual dose. Preferably, the individual dose contains an amount of the active drug that is administered one time, and typically corresponds to the total amount administered for one day, or ½, ⅓ or ¼ fold-amount thereof, but is not limited.

The human dose of the fermented tea extract can be suitably selected depending on the absorption, inactivation and excretion rates of the active ingredient in vivo, the patient's age, sex and conditions, and the severity of disease. For adults, the dose may be 10 to 300 mg/kg, preferably 20 to 100 mg/kg and may be administered 1 to 6 times a day, but is not limited.

Also, a composition according to one embodiment of the present invention may be used as a health food composition for preventing or ameliorating stroke, myocardial infarction, hypertension, hyperlipidemia, diabetes or obesity, which is formulated in the form of pills, capsules, tablets, granules or drinks.

The fermented tea extract of the present invention may be added to a health food. If the fermented tea extract of the present invention is used as a food additive, it may be added alone, or can be used in combination with other foods or food ingredients, or may be used appropriately according to other conventional methods. Mixed amounts of active ingredients may be suitably determined depending upon the purpose of use (prophylactic, health or therapeutic treatment). Generally, upon production of foods or beverages with which the fermented tea extract is mixed, the extract may be added preferably in an amount of 0.1 to 50 wt % based on the total weight of the food or beverage. However, when prolonged intake is intended for the purpose of health and hygiene or for health control, the amount of the fermented tea extract may be smaller than the lower limit of the above-specified range. In addition, even if the fermented tea extract is used in an amount larger than the upper limit of the above range, it does not cause a problem in terms of safety.

There is no particular limit to the kind of the health food composition. Examples of foods to which the fermented tea extract can be added include meats, sausages, bread, chocolate, candies, snack, confectionery, noodles, gum, dairy products including ice cream, various soups, beverages, teas, drinks, alcoholic beverages and multi-vitamin preparations. The health foods include all health foods in a conventional sense and are not limited to the above-mentioned foods.

A composition according to one embodiment of the present invention contains as an active ingredient the above-described fermented tea extract which reduces lipid levels in blood and the liver and inhibits vasoconstriction to induce vascular relaxation, thereby improving blood circulation.

Hereinafter, the present invention will be described in further detail with reference to examples. However, these examples are for illustrative purposes only and the scope of the present invention is not limited thereto.

### Example 1

### Preparation of Post-Fermented Tea

The strain Saccharomyces Cerevisiae was cultured in a shake incubator at 20˜40° C. for 72 hours. The culture was collected and centrifuged into the strain and the active medium. The strain was washed 2-4 times with 0.8-1.0% physiological saline, and then a fermentation broth consisting of a mixture of water and an energy source together with green tea was supplied thereto for suitable microbial metabolism. The fermentation broth was prepared by mixing, based on the total weight of the fermentation broth, 0.05-10.0 wt % of sugar and 0.005-10.0 wt % of fructose, sterilizing the mixture at a pressure of 27 psi (pounds per square inch) and a temperature of 120° C. for 15 minutes, cooling the sterilized mixture to room temperature (about 25° C.) and then adding 0.005-1.0 wt % of soybean powder thereto.

Also, for smooth fermentative metabolism of the strain damaged during the washing process, the strain washed 2-4 times with saline was mixed with 100-500 ml of a fermentation broth before adding soybean powder, and cultured in an incubator for 24 hours, thereby stabilizing the strain in the fermentation broth.

A strain stabilized in a fermentation broth containing no soybean powder was mixed with the fermentation broth containing the soybean powder protein to prepare a bacterial fermentation broth. In a sterilized reaction tank, the bacteria/fermentation broth mixture was added to a green tea matrix prepared in each small package unit such that the number of bacterial cells in the mixture was 10³-10⁸ CFU/ml. The ratio of the fermentation broth to the dried green tea leaves was 30-60 wt %, and the tea leaves were continuously stirred even after addition of the bacterial fermentation broth such that the internal temperature of the tea leaves was not rapidly increased, thereby preventing damage to the strain by a rapid increase in temperature. After 5-30 minutes, the reaction was completed, and the green tea/bacterial fermentation broth having reduced temperature was subjected to a fermentation process in a constant-temperature fermentation tank at a temperature of 20˜70° C. in a state in which the inlet of the tank was closed so as to prevent introduction of outdoor air. If the fermentation temperature is higher than 40° C., the growth of strains other than Bacillus spp will be difficult, and thus the proliferation of other various bacterial during the fermentation period can be inhibited. The fermentation process was carried out for 24 hours to 28 days, and the fermentation product was dried with hot air at a temperature of 80-120° C. for 5 hours.

The post-fermented tea prepared as described above has a total microbial account of 10²-10⁸ CFU/g or less which is within the standard range, and no pathogenic microorganisms were detected in the post-fermented tea.

### Example 2

### Preparation of Post-Fermented Tea Extract

1 kg of the post-fermented tea prepared in Example 1 was dipped in 5 L of 70% ethanol solution and refluxed at 80° C. for 3 hours, after which it was extracted at room temperature for 12 hours. The extract was filtered, concentrated under reduced pressure, and freeze-dried, thereby preparing a powder sample. The yield of the powder sample was 15-20%, and the prepared powder was stored at room temperature until use.

### Test Example 1

### Sensory Evaluation of Post-Fermented Tea

2 g of each of green tea, Pu-erh tea and the post-fermented tea was extracted in 100 ml of hot water at 75° C. (±1° C.) for 2 minutes, and the sensory evaluation thereof was performed by an expert panel of 10 persons. The overall preference, including water color (chromaticity, turbidity), fragrance (scent of grass, nutty fragrance, stale odor), and flavor (scorched smell, astringent taste, savory taste) was scored on a 9-point scale, and the results of the evaluation are shown in Table 1 below.

TABLE 1

| | Green tea | Pu-erh tea | Post-fermented tea |
|---|---|---|---|
| Water color (chromaticity, | 6.4 ± 0.52 | 2.1 ± 0.32 | 5.4 ± 0.27 |
| turbidity) | | | |
| Fragrance (scent of grass, | 5.8 ± 0.68 | 5.3 ± 0.71 | 7.1 ± 0.53 |
| nutty fragrance, stale odor) | | | |
| Flavor (scorched smell, as- | 5.0 ± 0.42 | 3.4 ± 0.12 | 6.8 ± 0.87 |
| tringent taste, savory taste) | | | |
| Overall preference | 6.0 ± 0.45 | 4.8 ± 0.45 | 7.6 ± 0.60 |

(very good: 9, good: 7, common: 5, bad: 3, very bad: 1)

As can be seen in Table 1 above, the scores of the taste (flavor) and fragrance of the post-fermented tea were significantly higher than those of green tea or Pu-erh tea, and the overall preference of the post-fermented tea was 1.6 points higher than that of green tea and 2.8 points higher than that of Pu-erh tea. This suggests that the taste and fragrance of the post-fermented tea according to one embodiment of the present invention were improved compared to those of conventional green tea and were superior to those of Pu-erh tea which is currently used as post-fermented tea.

### Test Example 2

### Measurement of the Ability of Post-Fermented Tea to Eliminate Free Radicals

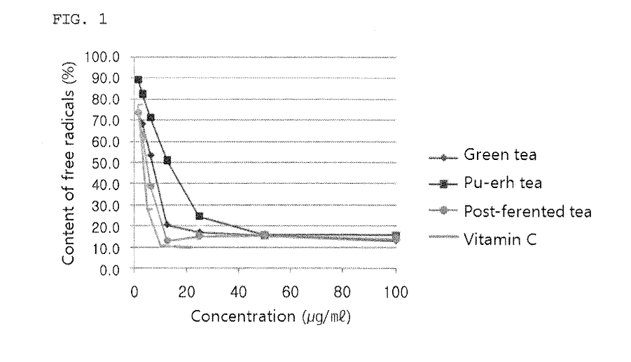

100 M of DPPH (Diphenyl Picryl Hydrazile) radicals were dissolved in 99% ethanol to prepare a radical solution. The test sample fermented tea prepared in Example 2 was dissolved in distilled water at various concentrations to prepare reaction solutions containing the radical solution and the test sample. As a control, a reaction solution not containing the test sample was used. The reaction solutions were sufficiently reacted at 37° C. for 30 minutes, and then the absorbance at 515 nm was measured, thereby determining the elimination of the radicals. As comparative groups, a green tea extract and a Pu-erh tea extract were used, and as a positive control group, vitamin C was used. The measurement results were calculated relative to 100 for the control group, and the calculation results are shown in FIG. 1.

As can be seen in FIG. 1, the post-fermented tea extract of the present invention eliminated the free radicals in a concentration-dependent manner. The effect of the post-fermented tea extract of the present invention on the elimination of the free radicals was similar to that of the positive control vitamin C and was about 10% higher than that of the comparative group green tea extract and 20-40% higher than that of the comparative group Pu-erh tea extract. This suggests that the post-fermented tea extract according to one embodiment of the present invention shows an excellent antioxidant effect.

### Test Example 3

### Observation of Vasoconstriction Inhibitory Effect Using Blood Vessel Ring

Experimental male white rats (Sprague-Dawley rats) weighing 250-300 g were supplied from Biolink (Seoul, Korea) and kept under a 12 hr light/12 hr dark cycle (7 a.m. and 7 p.m.) in animal facilities at a temperature of 22±2° C. and a humidity of 45-55%. Before use in the experiment, the animals were acclimated for one week while they were given feed (Purina Korea, Seoul, Korea) and water ad libitum.

The white rats were killed by blood loss, after which the chest was opened, and the thoracic aorta was rapidly dissected and immediately transferred into KR buffer (composition (mM); NaCl 115.5, KCl 4.6, KH₂PO₄ 1.2, MgSO₄ 1.2, CaCl₂ 2.5, NaHCO₃ 25.0, disodium. Ca²⁺ EDTA 0.026 mM, glucose 11.1, pH 7.4) saturated with a mixed gas of 95% O₂/5% CO₂. Blood and the surrounding fat and connective tissue were removed from the blood vessel, thus making blood vessel rings having a length of 3-4 mm. The blood vessel rings were tensioned gradually for the first 30 minutes to reach the equilibrium state, after which these were constricted with 10⁻⁶M phenylephrine and then relaxed by adding 10⁻⁶M acetylcholine thereto. Blood vessel rings showing a relaxation ratio of 80% or higher in the relaxation procedure were selected and used in the experiment. The buffer in the water bath was replaced with 90 mM KCl-containing KR (Krebs Ringer) buffer saturated with a mixed gas of 95% O₂/5% CO₂ so as to induce vasoconstriction which was used as the maximum constriction. The blood vessels were pretreated with the test material for 30 minutes, and then constriction-inducing phenylephrine was added thereto in a water bath at gradually increasing concentrations, and the resulting dose-response curve was observed. The vascular contractility was expressed relative to 100 for the vasoconstriction induced by 90 mM KCl, and the measurement results are shown in Table 2 below.

TABLE 2

| Phenyl- | | | | Post-fer- |
|---|---|---|---|---|
| ephrine | | Green tea | Pu-erh tea | mented tea |
| concentra- | Control (con- | extract (con- | extract (con- | extract (con- |
| tion (μM) | tractility %) | tractility %) | tractility %) | tractility %) |
| 0.1 | 22.36 ± 4.76 | 10.75 ± 3.2 | 15.32 ± 6.5 | 0.45 ± 1.1 |
| 1 | 61.95 ± 0.21 | 35.97 ± 5.52 | 32.48 ± 2.67 | 9.36 ± 2.82 |
| 10 | 90.20 ± 4.32 | 62.45 ± 9.04 | 58.73 ± 5.69 | 36.88 ± 5.13 |
| 100 | 98.79 ± 8.64 | 78.86 ± 1.68 | 87.69 ± 3.44 | 54.50 ± 13.67 |

As can be seen in Table 2 above, as the concentration of the constriction inducer phenylephrine increased, the vascular contractility increased. The effect of the post-fermented tea extract on the inhibition of vasoconstriction was 1.5-20 times higher than that of the control green tea extract depending on the concentration of phenylephrine. Thus, it could be observed that the post-fermented tea extract according to one embodiment of the present invention had a very excellent effect on the inhibition of vasoconstriction.

### Test Example 4

### Reduction in the Serum and Liver Lipid Levels in Animal Model

Eight 8-week-old female rats weighing 250-300 g were housed in each polycarbonate cage under a 12 hr light/12 hr dark cycle at a constant temperature of 22±2° C. and a relative humidity of 55±15%. The animals were fed with a normal diet and a high-cholesterol diet and given drinking water ad libitum. The high-cholesterol diet inducing hyperlipidemia has the composition shown in Table 3 below.

TABLE 3

| Components | Contents (%) |
|---|---|
| Sugar | 65.0 |
| Vitamin-free casein | 15.0 |
| AIN-76 mineral mixture | 3.5 |
| AIN-76 vitamin mixture | 1.0 |
| Corn oil | 2.0 |
| Lard | 8.0 |
| Cholesterol | 1.0 |
| Cholic acid | 0.5 |
| Alpha cell | 4.0 |
| Total | 100.0 |

The test animals were divided into four groups: a normal group, a control group, a positive control group, and a test group. The normal group was provided only with general feed, the control group was fed with a normal diet containing 1% cholesterol and 0.5% cholic acid to cause hyperlipidemia, and the positive control group was administered with fenofibric acid (200 mg/kg) that is used as an agent for treating hyperlipidemia, in which the fenofibric acid was suspended in 1% methyl cellulose (MC) and administered orally. The test group was administered orally with the post-fermented tea extract and the green tea extract every day together with a hyperlipidemia-inducing diet, in which the post-fermented tea extract and the green tea extract (200 mg/kg) were provided by oral administration once a day for 4 weeks. After the rats of each group were fasted for 12 hours, a blood sample was taken from the orbital vein and centrifuged at 10000 rcf for 10 minutes. The resulting serum was used to evaluate blood total cholesterol, LDL cholesterol and HDL-cholesterol levels. The rat's liver was collected and used to evaluate the liver cholesterol and triglyceride levels. The above analysis was carried out using an automatic blood analyzer and a Roche diagnostic kit, and the results of the analysis are shown in Table 4 (serum lipids) below and Table 5 (liver lipids).

TABLE 4
Serum lipids

| | | | | Test group | | |
|---|---|---|---|---|---|---|
| | Normal | Control | Positive control | | | Post- |
| Lipids (mg/dl) | group | group | group | Green tea | Pu-erh tea | fermented tea |
| Total cholesterol | 114.25 ± 2.41 | 218.57 ± 20.44 | 112.42 ± 13.33 | 132.49 ± 7.15 | 141.04 ± 10.32 | 136.25 ± 15.88 |
| HDL-cholesterol | 84.85 ± 0.35 | 55.72 ± 3.56 | 87.89 ± 6.84 | 58.84 ± 10.01 | 57.24 ± 6.31 | 59.43 ± 8.70 |
| LDL-cholesterol | 30.5 ± 6.42 | 134.75 ± 9.41 | 75.34 ± 2.80 | 8044 ± 8.43 | 87.32 ± 8.76 | 86.75 ± 2.51 |

As can be seen in Table 4 above, the serum total cholesterol level in the mice taken with the high-cholesterol feed for 4 weeks was about 2 times higher in the control group than in the normal group, and the LDL-cholesterol level was about 4.4 times higher in the control group, but the HDL-cholesterol level was reduced. These results indicate that the induction of hyperlipidemia by the hyperlipidemia-inducing diet was well performed. With respect to the test group administered with the post-fermented tea together with the hyperlipidemia-inducing diet, the total cholesterol level was 136.25 mg/dl which was about 38% lower than that in the control group, and the LDL-cholesterol level was about 36% lower than that in the control group, but the HDL-cholesterol level was not significantly increased. Meanwhile, the green tea extract showed decreases in total cholesterol level and LDL-cholesterol level of 39% and 41%, respectively, which were slightly higher than those obtained by the post-fermented tea extract. However, the Pu-erh extract showed decreases in total cholesterol level and LDL-cholesterol level of 36% and 35%, respectively, which were the lowest among the test materials.

TABLE 5
Liver lipids

| | | | Positive | Test group | | |
|---|---|---|---|---|---|---|
| Lipids | Normal | Control | control | | | Post- |
| (μg/mg) | group | group | group | Green tea | Pu-erh tea | fermented tea |
| Total cholesterol | 5.81 ± 0.85 | 27.96 ± 2.46 | 15.43 ± 0.52 | 20.46 ± 8.21 | 21.85 ± 0.94 | 18.77 ± 4.01 |
| Triglyceride | 199.91 ± 24.33 | 230.35 ± 10.53 | 185.39 ± 23.84 | 190.46 ± 5.69 | 186.72 ± 9.18 | 180.43 ± 16.73 |

As can be seen in Table 5 above, the liver total cholesterol and triglyceride levels in the rats of the control group were significantly higher than those in the normal group, indicating that the hyperlipidemia-inducing diet induced fatty liver. In the group treated with the green tea extract, the total cholesterol and triglyceride levels were reduced by 27% and 17%, respectively, and in the group treated with the Pu-erh tea extract, the total cholesterol and triglyceride levels were reduced by 22% and 19%, respectively, and in the group treated with the post-fermented tea, the total cholesterol and triglyceride levels were reduced by 33% and 22%, respectively. These results suggest that treatment with the post-fermented tea extract has the most excellent effect on a reduction in liver lipid levels.

The composition containing the post-fermented tea extract according to the present invention can be applied in various formulations as described below, but is not limited thereto.

### Preparation Example 1

### Soft Capsule Formulation

100 mg of the post-fermented tea extract, 50 mg of a soybean extract, 180 mg of soybean oil, 50 mg of a red ginseng extract, 2 mg of palm oil, 8 mg of hydrogenated palm oil, 4 mg of yellow beeswax and 6 mg of lecithin were mixed with each other, and 400 mg of the mixture was filled in each capsule according to a conventional method, thereby preparing a soft capsule.

### Preparation Example 2

### Tablet Formulation

100 mg of the post-extracted tea extract, 50 mg of a soybean extract, 100 mg of glucose, 50 mg of a red ginseng extract, 96 mg of starch and 4 mg of magnesium stearate were mixed with each other, 40 mg of 30% ethanol was added thereto, and the mixture was granulated. The granules were dried at 60° C. and compressed into a tablet using a tableting machine.

### Preparation Example 3

### Granule Formulation

100 mg of the post-fermented tea extract, 50 mg of a soybean extract, 100 mg of glucose, 50 mg of a red ginseng extract and 600 mg of starch were mixed with each other, 100 mg of 30% ethanol was added thereto, and the mixture was dried at 60° C., granulated and then filled in a sachet. The final weight of the content of the sachet was 1 g.

### Preparation Example 4

### Drink Formulation

100 mg of the post-fermented tea extract, 50 mg of a soybean extract, 10 g of glucose, 50 mg of a red ginseng extract, 2 g of citric acid and 187.8 g of purified water were mixed with each other and filled in a bottle. The final volume of the content of the bottle was 200 ml.

1. A composition for improving blood circulation, which contains, as an active ingredient, a fermented tea extract obtained by inoculating tea leaves with yeast as a fermentation strain and fermenting the inoculated tea leaves.

2. The composition of claim 1, wherein the fermentation strain is one or more selected from the group consisting of Saccharomyces Carsbergensis, Saccharomyces Sake, Saccharomyces Ellipsoideus, Saccharomyces Coreanus, and Saccharomyces Cerevisiae.

3. The composition of claim 1, wherein the fermentation strain is Saccharomyces Cerevisiae.

4. The composition of claim 1, wherein the fermented tea extract is contained in an amount of 0.1-50 wt % based on the total weight of the composition.

5. A pharmaceutical composition for preventing or treating stroke, myocardial infarction, hypertension, hyperlipidemia, diabetes or obesity, which contains, as an active ingredient, a fermented tea extract obtained by inoculating tea leaves with yeast as a fermentation strain and fermenting the inoculated tea leaves.

6. The pharmaceutical composition of claim 5, wherein the fermentation strain is one or more selected from the group consisting of Saccharomyces Carsbergensis, Saccharomyces Sake, Saccharomyces Ellipsoideus, Saccharomyces Coreanus, and Saccharomyces Cerevisiae.

7. The pharmaceutical composition of claim 5, wherein the fermentation strain is Saccharomyces Cerevisiae.

8. The pharmaceutical composition of claim 5, wherein the fermented tea extract is contained in an amount of 0.1-50 wt % based on the total weight of the composition.

9. A health food composition for preventing or ameliorating stroke, myocardial infarction, hypertension, hyperlipidemia, diabetes or obesity, which contains, as an active ingredient, a fermented tea extract obtained by inoculating tea leaves with yeast as a fermentation strain and fermenting the inoculated tea leaves.

10. The health food composition of claim 9, wherein the fermentation strain is one or more selected from the group consisting of Saccharomyces Carsbergensis, Saccharomyces Sake, Saccharomyces Ellipsoideus, Saccharomyces Coreanus, and Saccharomyces Cerevisiae.

11. The health food composition of claim 9, wherein the fermentation strain is Saccharomyces Cerevisiae.

12. The health food composition of claim 9, wherein the fermented tea extract is contained in an amount of 0.1-50 wt % based on the total weight of the composition.
